(19) United States
(12) Patent Application Publication
(10) Pub. No.: US 2025/0342950 A1
(43) Pub. Date: Nov. 6, 2025

(54) VOLUMETRIC ESTIMATION OF HEALTH CARE INVENTORY

(71) Applicant: TaskUnite Inc., Buffalo, NY (US)

(72) Inventors: Bianca Gonzalez, Buffalo, NY (US); Ken Chhan, Los Angeles, CA (US)

(21) Appl. No.: 19/200,255

(22) Filed: May 6, 2025

Related U.S. Application Data
(60) Provisional application No. 63/643,194, filed on May 6, 2024.

(51) Int. Cl. G16H 40/40; G06T 7/10; G16H 30/40; G16H 40/63

(52) U.S. Cl. CPC G16H 40/40; G06T 7/10; G16H 30/40; G16H 40/63; G06T 2207/20081; G06T 2207/30176; G06T 2207/30242

(57) ABSTRACT

A system may detect a triggering condition by an imaging device. The system may capture, by the imaging device in response to the triggering condition, an image of a container. The system may perform, by a machine learning model, pixel quantification of the container. The system may determine, based on the pixel quantification, a status of the container.

### CROSS REFERENCE TO RELATED APPLICATION(S)

This application claims priority to and the benefit of U.S. Provisional Application Patent Ser. No. 63/643,194, filed May 6, 2024, the entire disclosures of which are herein incorporated by reference.

### TECHNICAL FIELD

This disclosure relates to a health care inventory imaging and tracking intelligence system, in particular, to a system for volumetric estimation to monitor, track, and analyze inventory usage in the health care field based on imaging, motion, and/or other detections.

### BACKGROUND

Inefficiencies in health care inventory tracking and supply chain management contribute to significant operational and financial burdens within health care environments. These inefficiencies often stem from reliance on manual processes or limited-use automation that fails to provide real-time insights into inventory conditions. Health care providers may experience issues such as inventory leakage, expired or misplaced stock, inaccurate counts, missed restocking events, and excessive administrative overhead related to manual auditing or reordering.

Traditional systems may not account for the dynamic nature of inventory consumption during patient care and staff workflows. As a result, inventory usage tied to specific procedures, departments, or patient interactions often goes undocumented. This lack of visibility hinders the ability of health care institutions to perform meaningful analysis related to consumption trends, task-based cost attribution, and resource optimization.

Further, the absence of real-time volumetric data makes it difficult for health care providers to maintain optimal inventory levels. Without visibility into current inventory volumes or trends in usage, staff must rely on fixed reorder schedules or reactive replenishment, both of which may lead to shortages or overstocking. These gaps impact not only operational efficiency but also patient care and regulatory compliance.

### SUMMARY

Disclosed herein are, inter alia, implementations of systems and techniques for volumetric estimation of health care inventory.

In one implementation, a method includes detecting a triggering condition by an imaging device, capturing, by the imaging device in response to the triggering condition, an image of a container, performing, by a machine learning model, pixel quantification of the container, and determining, based on the pixel quantification, a status of the container.

In another implementation, a system includes an imaging device configured to detect a triggering condition and capture, in response to the triggering condition, an image of a container. The system further includes a server device in communication with the imaging device. The server device includes a processor configured to receive, from the imaging device, the image of the container, perform, by a machine learning model, pixel quantification of the container, and determine, based on the pixel quantification, a status of the container.

In yet another implementation, a method includes obtaining a set of training images including images of a container associated with inventory, wherein each training image is labeled with a status of the container, training, using the set of training images, a machine learning model to perform pixel quantification and associate pixel distributions with a corresponding status of the container, obtaining a new image of the container, performing, by the machine learning model for the container in the new image, pixel quantification of the container, and determining, by the machine learning model, the status of the container in the new image.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram showing an example of a real-time health care inventory imaging and tracking intelligence system.

FIG. 2 is a block diagram showing an example of an imaging and tracking device used in a real-time health care inventory imaging and tracking intelligence system.

FIG. 3 is a block diagram showing an example of an imaging and tracking device coupled to a furniture unit for monitoring and tracking inventory items.

FIG. 4 is a block diagram showing an example of a workflow of an intelligent vision system.

FIG. 5 is a block diagram showing an example of a workflow of a machine learning model for pixel quantification.

FIGS. 6A and 6B are illustrations showing examples of images of a containerized environment.

FIG. 7 is a flowchart showing an example of a technique for determining inventory using an intelligent vision system.

FIG. 8 is a flowchart showing an example of a technique for training a machine learning model for pixel quantification.

FIG. 9 is a block diagram showing an example of a computing device which may be used in a region-based electrical intelligence system.

### DETAILED DESCRIPTION

Implementations of this disclosure include using an intelligent vision system to estimate a status of a container (i.e., to track inventory volume). As used herein, tracking inventory volume refers to the automated process of monitoring and estimating the quantity of inventory items within a container by analyzing images to determine the fill status (e.g., full, partially full, empty) based on the proportion of inventory item pixels relative to container pixels. The container may be used to store health care inventory items. A system may include an imaging device configured to capture images of a containerized environment, where each container may include one or more inventory items. The imaging device may detect a triggering condition, such as motion or a time-based interval, and in response, capture an image of at least one container. The image may be transmitted to a server device that executes a machine learning model to perform pixel quantification on the container. In some implementations, the machine learning model may be implemented by the imaging device. The machine learning model may analyze pixel-level distributions within the container image to estimate a fill status of the container. The status may include an indication of whether the container is full, partially full, or empty. The estimated status may be stored in a database or used to initiate actions such as alerting or reordering.

The intelligent vision system may use a machine learning model trained on labeled images to identify visual features within the container image and classify pixel regions into categories, for example, inventory item pixels or container pixels. That is, the intelligent vision system may use a machine learning model trained on labeled images to identify visual features within the container image and classify pixels within pixel regions as, for example, inventory item pixels or container pixels, thereby determining the status of the container based on the proportion of these classified pixels.

The fill status of the container may be estimated based on the proportion of inventory item pixels relative to the total pixels in a defined region of interest. A region of interest refers to an area of the image, such as a bounding box or segmented container area, automatically defined (e.g., identified) by the intelligent vision system based on visual features or training data. In some implementations, label pixels may also be identified to allow for classifying the type of inventory item. The training process for the machine learning model may include examples of containers in different known fill states, and the model may apply the learned relationships during inference. In real-time operation, this enables the intelligent vision system to track inventory volume without requiring physical retrieval or manual inspection. The data generated by the intelligent vision system may be used to update a dashboard, notify clinical staff, reorder inventory, or maintain a record of inventory levels over time.

The intelligent vision system improves upon traditional inventory tracking technologies by enabling non-intrusive, automated, and real-time estimation of inventory volumes. Unlike systems that rely solely on physical withdrawal detection or manual counts, the disclosed intelligent vision system uses computer vision and machine learning to provide continuous inventory awareness. The intelligent vision system may use trained models, customized image processing techniques, and component interactions that are designed to operate together in a technical environment. For example, the intelligent vision system may distinguish between container pixels and inventory item pixels using learned pixel distributions and bounding box analysis. These functions may be performed in real time based on data received from physical sensors and computing components that perform image acquisition, model inference, and system response. The intelligent vision system enables pixel-level inventory tracking without requiring traditional inventory access methods such as manual scanning or weighing. Through the combination of trained image analysis, decision logic, and automated outputs, the intelligent vision system may enhance inventory visibility, reduce manual workload, and provide a more intelligent, adaptive inventory control process in health care and other regulated settings.

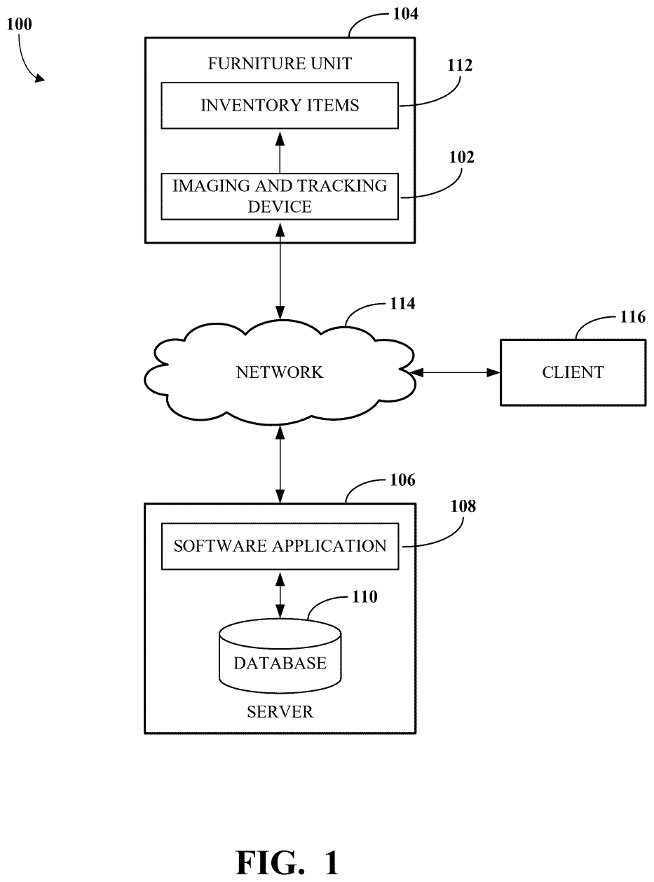

To describe some implementations in greater detail, reference is first made to examples of hardware and software structures used to implement a real-time health care inventory imaging and tracking intelligence system. FIG. 1 is a block diagram showing an example of a real-time health care inventory imaging and tracking intelligence system 100. The system 100 includes an imaging and tracking device 102 coupled to a furniture unit 104 and a server 106 that runs a software application 108 and stores a database 110.

The imaging and tracking device 102 is a device which is used to monitor inventory items 112 stored within or on the furniture unit 104. The furniture unit 104 is or includes a piece of furniture with at least one surface configured for storing the inventory items 112. The inventory items 112 may be stored within individual containers (e.g., bins, housings, storage units, or the like). In some implementations, the furniture unit 104 may include a number of shelves of the same or different sizes. In some implementations, the furniture unit 104 may include a number of drawers of the same or different sizes. In some implementations, the furniture unit may include a number of cabinets of the same or different sizes. In some implementations, the furniture unit 104 may include a combination of shelves, drawers, and/or cabinets. The furniture unit 104 may be configured to store the inventory items 112 at particular temperatures. For example, the furniture unit 104 may be a refrigerated unit. In another example, the furniture unit 104 may be a heated unit. It will be understood that, aside from the foregoing examples and implementations, the furniture unit 104 may include other types of open or enclosed surfaces or sets of surfaces within or upon which the inventory items 112 may be stored. For example, the furniture unit 104 may be a closet, a freestanding shelving unit, a mobile supply cart, or another structure designed to organize or store the inventory items 112. In some implementations, the furniture unit 104 may include one or more enclosures (e.g., a door, a lid, a sliding panel, a curtain, or the like) to protect or conceal the inventory items 112.

The inventory items 112 are items that may be used to provide health care support to a patient. Examples of the inventory items 112 include, but are not limited to, bandages, gauze materials, syringes, medication bottles, ointments, needles, intravenous delivery mechanisms, fluids, medical tapes, and other materials. The inventory items 112 are stored within or on the furniture unit 104. For example, where the furniture unit 104 is a shelving unit with a number of shelves, each shelf of the furniture unit 104 can store some of the inventory items 112. In another example, some of the inventory items 112 may be stored on some of the shelves of the furniture unit 104, while other shelves of the furniture unit 104 do not store inventory items 112.

The imaging and tracking device 102 includes an image sensor, a processing component configured to process data captured using the image sensor, a network interface for communicating information processed using the processing component to other devices (e.g., the server 106), and a power source for supplying power for use by the image sensor, the processing component, and the network interface. The imaging and tracking device 102 monitors activity occurring with respect to the furniture unit 104, such as to detect when an inventory item of the inventory items 112 is removed from the furniture unit 104 and to identify the inventory item that was removed. In some implementations, the imaging and tracking device 102 may use sensors other than an image sensor to detect and identify removed inventory items of the inventory items 112. For example, the imaging and tracking device 102 may include a motion sensor. In another example, the imaging and tracking device 102 include an accelerometer or other sensor capable of detecting vibrations to which the furniture unit 104 is exposed. In yet another example, the imaging and tracking device 102 may include another sensor usable to detect changes within the furniture unit 104.

The imaging and tracking device 102 may be removably coupled to a portion of the furniture unit 104. For example, the imaging and tracking device 102 may be coupled to a portion of the furniture unit 104 using a hook and loop fastener, an adhesive strip, a mounting mechanism which enables the removal of the imaging and tracking device 102 from the furniture unit 104, or another removable coupling technique. Alternatively, the imaging and tracking device 102 may be permanently coupled to a portion of the furniture unit 104. For example, the imaging and tracking device 102 may be installed using screws or other mechanical fasteners, an adhesive, a mounting mechanism which prevents the removal of the imaging and tracking device 102 from the furniture unit 104, or another permanent coupling technique. The imaging and tracking device 102 may be mounted in an area surrounding the furniture unit 104 (e.g., a wall near the furniture unit 104, a door enclosing the furniture unit 104, a nearby furniture unit 104, or the like) or may be mounted to different areas of the furniture unit 104 (e.g., a shelf, a wall, a door, a lid, or another structural feature).

The server 106 is a computing aspect that runs the software application 108. The server 106 may be or include a hardware server (e.g., a server device), a software server (e.g., a web server and/or a virtual server), or both. For example, where the server 106 is or includes a hardware server, the server 106 may be a server device located in a rack, such as of a data center.

The software application 108 is used to process information received from the imaging and tracking device 102, for example, over a network 114. In some implementations, the software application 108 can be used to process information received from the imaging and tracking device 102 to identify an inventory item of the inventory items 112 which has been physically retrieved from the furniture unit 104. In some implementations, the software application 108 can be used to update database records associated with retrieved inventory items from the inventory items 112. In some implementations, the software application 108 can be used to transmit signals indicative of updated database records to a client 116. In some implementations, the software application 108 is a web application run within a web page served by server 106 and accessed, for example, by the client 116. In some implementations, the software application 108 is a mobile application which includes a server-side application running on the server 106 and a client-side application running on the client 116.

The software application 108 accesses the database 110 stored on the server 106 to perform at least some of the functionality of the software application 108. The database 110 is a database or other data store used to store, manage, or otherwise provide data used to deliver functionality of the software application 108. The database 110 may, for example, be a relational database management system, an object database, an XML database, a configuration management database, a management information base, one or more flat files, other suitable non-transient storage mechanisms, or a combination thereof.

The database 110 can store records relating to inventory supplies (e.g., the inventory items 112) which are or may be monitored using the imaging and tracking device 102 or by a different imaging and tracking device within the furniture unit 104 or within a different furniture unit. The database 110 can also store records relating to the usage, including pre-care and post-care instructions, for some or all of the inventory items 112. The database 110 can also store records related to administrative tasks, patient-related tasks, patient names, staff members authorized to retrieve the inventory items 112 from the furniture unit 104, and/or other records.

The software application 108 includes a dashboard which enables a user thereof (e.g., a user of the server 106 or a user of the client 116) to review information processed using the system 100. For example, the dashboard can be used to review information received at the software application 108 from the imaging and tracking device 102. In another example, the dashboard can be used to review changes made to records within the database 110 based on the information received from the imaging and tracking device 102. In yet another example, the dashboard can be used to view information (e.g., knowledgebase articles or the like) associated with inventory items 112 which have been detected as being physically retrieved from the furniture unit 104.

The imaging and tracking device 102 communicates with the server 106 over the network 114. The network 114 may, for example, be a local area network, a wide area network, a machine-to-machine network, a virtual private network, or another public or private network. Communication over the network 114 may use one or more network protocols, such as using Ethernet, TCP, IP, power line communication, Wi-Fi, Bluetooth®, infrared, GPRS, GSM, CDMA, Z-Wave, ZigBee, another protocol, or a combination thereof.

The client 116 may be given access to the software application 108. The client 116 may be or include a hardware client (e.g., a client device), a software client (e.g., a web server and/or a virtual server), or both. For example, the client 116 may be a mobile device, such as a smart phone, tablet, laptop, or the like. In another example, the client 116 may be a desktop computer or another non-mobile computer. The client 116 may run a client-side software application or other software to communicate with the software application 108. For example, the client-side software application may be a mobile application that enables access to some or all functionality and/or data of the software application 108. The client 116 communicates with the server 106 over the network 114.

Implementations of the real-time health care inventory imaging and tracking intelligence system 100 may differ from what is shown and described with respect to FIG. 1. In some implementations, the imaging and tracking device 102 communicates with the server 106 over the network 114 using an intermediary relay. For example, the intermediary relay may be or include network hardware, such as a router, a switch, a load balancer, another network device, or a combination thereof. The intermediary relay may receive information and/or commands from and/or transmit information and/or commands to the imaging and tracking device 102 using one or more network protocols, such as using Ethernet, TCP, IP, power line communication, Wi-Fi, Bluetooth®, infrared, GPRS, GSM, CDMA, Z-Wave, ZigBee, another protocol, or a combination thereof.

In some implementations, the server 106 and the client 116 may each represent computing devices located within a common area. For example, the server 106 and the client 116 may both be computers located within a health care clinic or hospital. In some implementations, the server 106 and the client 116 may be combined into a single computing device. In some implementations, the software application 108 may transmit push notifications, text messages, or other alerts to client 116 without client 116 first accessing the software application 108 (e.g., via a webpage or otherwise). For example, the software application 108 can be configured to automatically transmit signals to certain clients, such as using a whitelist or otherwise.

In some implementations, a health care facility may use multiple imaging and tracking devices. For example, each of the multiple imaging and tracking devices may be coupled to a different furniture unit or to different shelves, drawers, or cabinets of the same furniture unit. The software application 108 can be used to receive and process signals from each of the multiple imaging and tracking devices. For example, the software application 108 can identify individual imaging and tracking devices from which data is received, such as within a graphical user interface (GUI) generated by the software application 108 based on the retrieval of an inventory item of the inventory items 112.

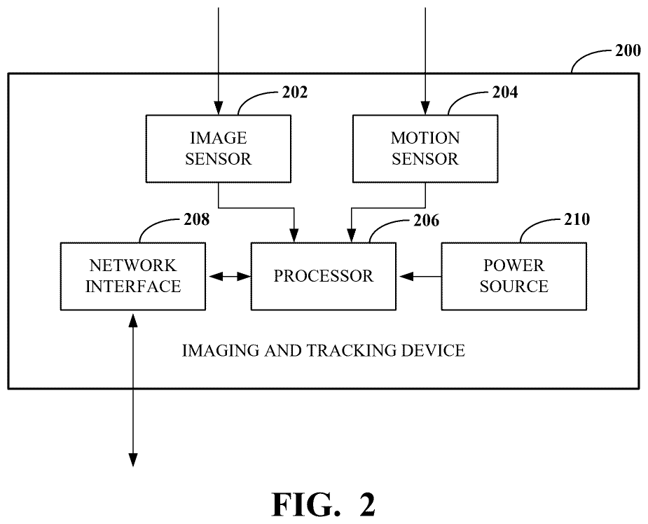

FIG. 2 is a block diagram showing an example of an imaging and tracking device 200 used in a real-time health care inventory imaging and tracking intelligence system, for example, the system 100 shown in FIG. 1. For example, the imaging and tracking device 200 may be the imaging and tracking device 102 shown in FIG. 1. The imaging and tracking device 200 includes an image sensor 202, a motion sensor 204, a processor 206, a network interface 208, and a power source 210.

The image sensor 202 is a sensor configured to capture images within a field of view of the image sensor 202 or otherwise capture data used to construct images. The image sensor 202 may, for example, be a charge-coupled device sensor, an active pixel sensor, a complementary metal-oxide semiconductor sensor, an N-type metal-oxide-semiconductor sensor, or another sensor or combination of sensors.

The motion sensor 204 is a sensor configured to detect motion within a field of motion of the motion sensor 204. The motion sensor 204 may, for example, be an infrared sensor (e.g., a passive infrared sensor), a microwave sensor, an area reflective sensor, an ultrasonic sensor, or another sensor or combination of sensors.

The processor 206 is a central processing unit, such as a microprocessor, and can include single or multiple processors having single or multiple processing cores. In some implementations, the processor 206 may be or otherwise refer to an integrated circuit, for example, a field programmable gate array (e.g., FPGA), programmable logic device (PLD), reconfigurable computer fabric (RCF), system on a chip (SoC), an application specific integrated circuit (ASIC), and/or another type of integrated circuit. The processor 206 includes a cache, or cache memory, for local storage of operating data and/or instructions. For example, the cache can be used to temporarily store data recorded using the image sensor 202, the motion sensor 204, and/or another sensor (e.g., in implementations in which the imaging and tracking device 200 includes such another sensor, such as described below).

The network interface 208 is used to transmit information and/or commands to and/or receive information and/or commands from one or more devices external to the imaging and tracking device 200. The network interface 208 provides a connection or link to a network (e.g., the network 114 shown in FIG. 1). The network interface 208 can be a wired network interface or a wireless network interface. The imaging and tracking device 200 can communicate with other devices via the network interface 208 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

The power source 210 is a source for providing power to the imaging and tracking device 200. For example, the power source 210 can be an interface to an external power distribution system. In another example, the power source 210 can be a battery, such as a coin-cell battery or another battery.

Implementations of the imaging and tracking device 200 may differ from what is shown and described with respect to FIG. 2. In some implementations, the motion sensor 204 may be omitted. In some implementations, one or more other sensors may be included. For example, in some such implementations, the imaging and tracking device 200 may include an accelerometer or other sensor capable of detecting vibrations. The accelerometer or other sensor may be used to monitor for vibrations (e.g., indicative of a person accessing a furniture unit to which the imaging and tracking device 200 is coupled, such as by the person opening a door or pulling on a drawer or shelf of the furniture unit).

The imaging and tracking device 200 may operate in a wait state or an active state to optimize resource usage, such as battery power. In the wait state, the processor 206 restricts image capture by the image sensor 202 to preserve resources. The processor 206 monitors sensor measurements from one or more other sensors, such as an accelerometer detecting vibrations at the furniture unit 104. At a given time, the processor 206 determines whether the sensor measurements, representative of vibration intensity or frequency, meet a predefined threshold indicative of accessing the furniture unit. If the measurements do not meet the threshold, the processor 206 determines to maintain the imaging and tracking device 200 in the wait state. If the measurements meet the threshold, the processor 206 changes the device to the active state, enabling image capture by the image sensor 202.

In the active state, the image sensor 202 captures images of inventory items within the furniture unit 104, such as a first image at a first time. At a second time after the first time, the one or more other sensors produce first sensor measurements representative of vibrations at the furniture unit 104. If the processor 206 determines these measurements do not meet the threshold, the imaging and tracking device 200 remains in the wait state, preserving resources by restricting image capture. At a third time after the second time, the sensors produce second sensor measurements. If the processor 206 determines these measurements meet the threshold, the device transitions to the active state, and the image sensor 202 captures a second image of the inventory items. The processor 206 may process these images to detect visual characteristics, such as color or shape, to identify and enumerate subsets of inventory items by type, enabling precise inventory tracking.

In some implementations, the processor 206 may be configured to initiate image capture by the image sensor 202 based on a predefined schedule, such as at regular time intervals (e.g., every hour or shift). This allows the imaging and tracking device 200 to periodically monitor inventory without requiring a motion or vibration trigger, supporting consistent tracking of container fill status.

The implementation of the imaging and tracking device 200 shown in FIG. 2 includes each of the image sensor 202, the motion sensor 204, the processor 206, the network interface 208, and the power source 210 as being included within a single housing or other enclosure. However, in some implementations, the components of the imaging and tracking device 200 may be physically separated into multiple housings or other enclosures, or otherwise separated. For example, in some such implementations, the image sensor 202 and the motion sensor 204 may be included in a first portion of the imaging and tracking device 200 and the processor 206 and the network interface 208 may be included in a second portion of the imaging and tracking device 200. The first portion may be coupled to the furniture unit. The second portion may be external to the furniture unit.

In some implementations, the power source 210 can cause the network interface 208 to transmit a signal indicating a low power status of the imaging and tracking device 200. For example, a server device running a software application (e.g., the server 106 and the software application 108 shown in FIG. 1) can receive a signal indicating a low power status of the imaging and tracking device 200. The software application can then indicate the low power status, such as to one or more client devices of personnel of the health care provider that uses the imaging and tracking device 200.

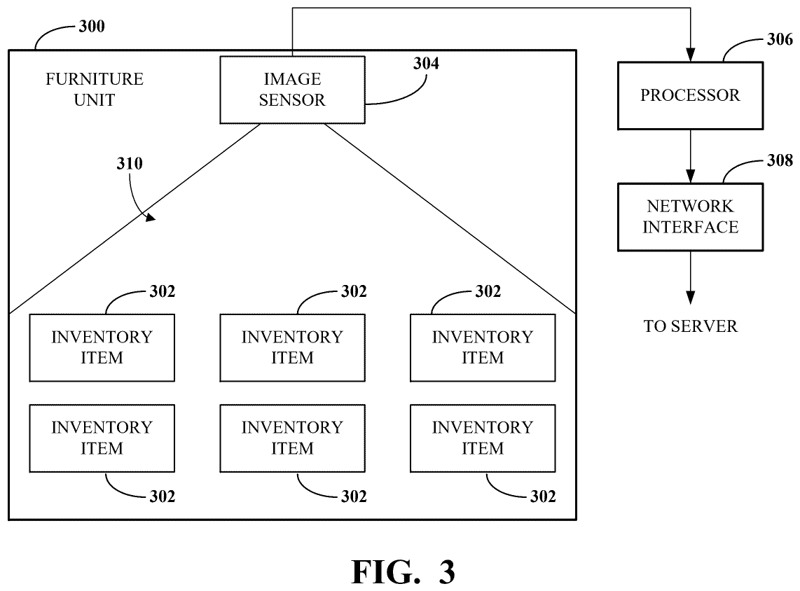

FIG. 3 is a block diagram showing an example of an imaging and tracking device coupled to a furniture unit 300 for monitoring and tracking inventory items 302. In particular, an image sensor 304, a processor 306, and a network interface 308 of the imaging and tracking device are shown. The image sensor 304, the processor 306, and the network interface 308 may, for example, respectively be the image sensor 202, the processor 206, and the network interface 208 shown in FIG. 2. In the implementation shown in FIG. 3, the image sensor 304 is coupled to the furniture unit 300, and the processor 306 and the network interface 308 are external to the furniture unit 300.

The image sensor 304 has a field of view 310. The field of view 310 represents the physical area of the furniture unit 300 for which the image sensor 304 is configured to capture images. In some implementations, the field of view 310 may be adjustable, such as by selectively opening or narrowing aspects of the image sensor. In some implementations, the image sensor may be included in a controllable mechanism. For example, a user of a software application that processes information received from the imaging and tracking device may use the software application to remotely control, in real-time, a direction of the image sensor 304. Changing the direction of the image sensor 304 causes the specific location of the field of view 310 to change.

Implementations of the imaging and tracking device may differ from what is shown and described with respect to FIG. 3. In some implementations, one or more sensors external to the image sensor 304 may be coupled to the furniture unit 300. For example, the one or more sensors may include weight or pressure sensors configured to detect differences in an amount of weight or pressure applied to a surface on which the inventory items 302 are stored. Such a weight or pressure sensor can be used to detect the physical retrieval of one or more of the inventory items 302. For example, the data recorded using such a weight or pressure sensor can be processed by the processor 306 to detect the physical retrieval of an inventory item 302. In some such implementations, a single weight or pressure sensor may be configured to measure changes in weight or pressure for the entire surface of the furniture unit 300. In other such implementations, multiple weight or pressure sensors may each be disposed in a different location about the surface of the furniture unit 300 and configured to measure changes in weight or pressure for their specific locations.

In some implementations, a light source may be used to illuminate all or a portion of the furniture unit 300. For example, where the furniture unit 300 is or includes an enclosed piece of furniture, the image sensor 304 may not be exposed to enough light to effectively capture images for detecting retrievals of the inventory items 302. In some such implementations, the image sensor 304 and the light source may be included in a common housing or other enclosure.

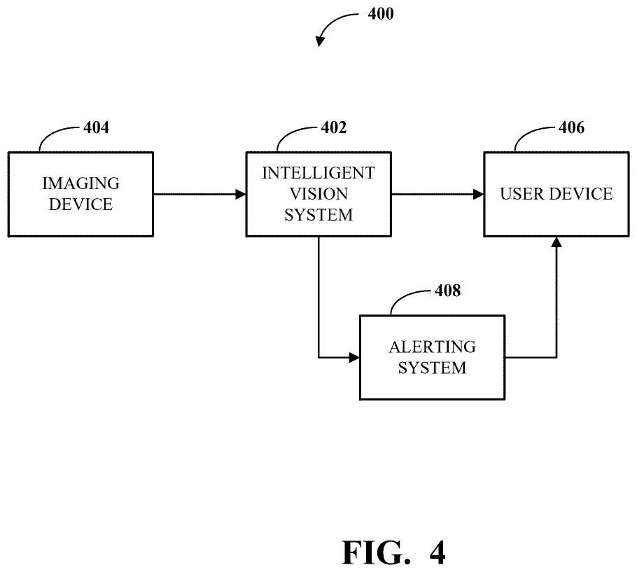

FIG. 4 is a block diagram showing an example of a first workflow 400 of an intelligent vision system 402. The intelligent vision system 402 may include a processor configured to perform pixel quantification of at least one container based on image data. The intelligent vision system 402 may be configured to perform pixel quantification using a machine learning model. The machine learning model may be trained on a set of training images labeled with a status of the container. The intelligent vision system 402 may determine a status of the container, for example, a fill status of the container (e.g., full, partially full, or empty). In some implementations, the fill status of the container may be a percentage, for example, 0% full, 20% full, 50% full, 90% full, 100% full, or the like.

Pixel quantification, as used herein, refers to analyzing a container image by segmenting and classifying pixels into categories, such as inventory item pixels, container pixels, or, in some implementations, label pixels (e.g., for labels affixed to the container), and calculating the proportion of inventory item pixels to container pixels within a defined region, such as a bounding box, to estimate the fill status of the container.

The intelligent vision system 402 may receive images from an imaging device 404 (e.g., the imaging and tracking device 200 shown in FIG. 2). The imaging device 404 may be configured to detect a triggering condition. The triggering condition may include motion detection, vibration detection, infrared motion detection, or expiration of a predefined time interval. The imaging device 404 may be configured to ignore the triggering condition based on specific instructions. For example, the imaging device 404 may be configured to ignore the triggering condition due to manual override, instructions to ignore triggering conditions within a predefined time period after a triggering condition, or the like. The imaging device 404 may be configured to receive (e.g., from the intelligent vision system 402) an updated triggering condition (e.g., an updated threshold or updated time interval). The imaging device 404 may replace the triggering condition with the updated triggering condition, and may transmit (e.g., to the intelligent vision system 402) confirmation of the updated triggering condition.

The imaging device 404 may be configured to capture an image of at least one container in a containerized environment. Capturing an image of the at least one container may be performed in response to detecting the triggering condition. The imaging device 404 may include one or more sensors, such as an image sensor (e.g., the image sensor 202) or a motion sensor (e.g., the motion sensor 204), an infrared sensor, a vibration sensor, or the like. The imaging device 404 may be removably or permanently coupled to a furniture unit. The captured image may be transmitted from the imaging device 404 to the intelligent vision system 402. Transmitting the captured image from the imaging device 404 to the intelligent vision system 402 may include using a network interface of the imaging device 404 to wirelessly communicate the captured image to the intelligent vision system 402. The captured image may be communicated over a short-range communication protocol, for example, Wi-Fi or Bluetooth® Alternatively, the captured image may be communicated over a long-range, for example, via a cloud network or the internet.

In some implementations, the imaging device 404 may use different states for power preservation. For example, a wait state may be used to cause a power source of the imaging device 404 to preserve power, such as by causing a processor of the imaging device 404 to put the imaging device 404 into a low-power mode. In another example, an active state may be used to cause the power source to use necessary power to enable the other components of the imaging device 404 to detect a triggering condition.

The intelligent vision system 402 may communicate with a user device 406. The intelligent vision system 402 may communicate with the user device 406 over a short-range communication protocol. In some implementations, the short-range communication protocol can be Wi-Fi or Bluetooth®. Alternatively, The user device 406 may be configured to receive the status of the container from the intelligent vision system 402. The user device 406 may display the container status to a user. The displayed status may include whether the container is full, partially full, or empty. The user device 406 may be a mobile device or a desktop device. The user device 406 may run a client-side application for viewing inventory data.

The intelligent vision system 402 may be communicatively connected to an alerting system 408. The alerting system 408 may be configured to generate an alert based on the status of the container. The alert may be generated when the status of the container meets or falls below a predefined threshold condition. For example, the alert may be generated when the status changes from full to partially full, when the status becomes empty, or when the status falls below a specified percentage (e.g., 50%, 20%, 0%, or the like). The alerting system 408 may transmit the alert to the user device 406. The alert may notify a user associated with the user device 406 that the container requires replenishment or that an inventory condition has been met. The alert may be delivered as a push notification, text message, or interface prompt within the client application.

Alternatively, the alert may trigger a signal to an inventory management system to automatically place an order for the inventory item associated with the container. The signal may include information such as an item identifier, a quantity to be ordered, a location of the container, and an urgency level. The inventory management system may process the signal to generate a purchase order, initiate fulfillment with a supplier, or schedule internal restocking procedures. The system may apply predefined ordering rules based on consumption patterns, threshold levels, or supply chain constraints. In some implementations, user approval may be required to confirm the order before execution. Alternatively, the order may be placed automatically without manual intervention. Automatic inventory ordering may reduce manual inventory tracking and help maintain optimal stock levels in health care environments.

In some implementations, the intelligent vision system 402 may generate an output comprising an image of the containerized environment annotated with fill status labels for remote viewing by an authorized user through an administrative user interface. To illustrate, upon a user request for inventory status via the administrative user interface, the imaging device 404 captures an image or video feed of the containers within the furniture unit. The intelligent vision system 402 processes the image using the machine learning model to determine the fill status (e.g., full, partially full, empty) of each container based on pixel quantification, as described herein. The intelligent vision system 402 then annotates each container in the image with a visual label indicating its fill status, such as a green label for full, yellow for partially full, or red for empty, typically positioned in a designated region (e.g., top left corner) of the container's bounding box.

Authorized users, such as clinical staff or inventory managers, may access the administrative user interface via a client device (e.g., client 116) to remotely monitor inventory. The user interface may display the annotated image, enabling users to visually assess the fill status of multiple containers simultaneously. The annotated labels facilitate rapid identification of inventory conditions, supporting timely restocking decisions or compliance checks. The system may store the annotated image in the database (e.g., database 110) for record-keeping or transmit it to the user device as part of an alert or status update, enhancing inventory visibility and operational efficiency in healthcare settings.

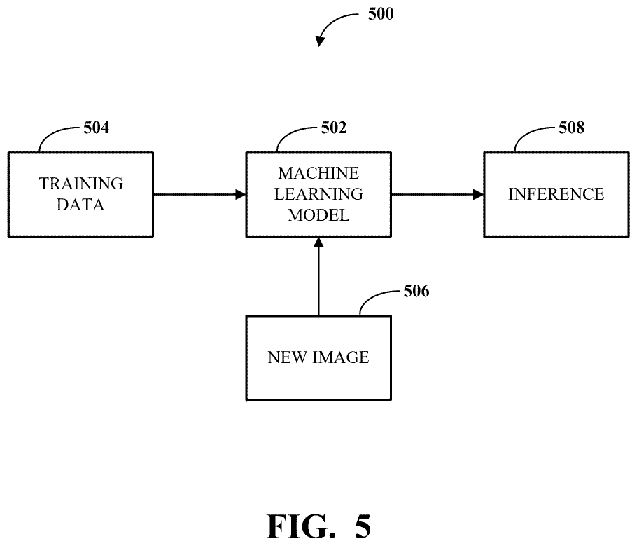

FIG. 5 is a block diagram showing an example of a second workflow 500 of a machine learning model 502 for pixel quantification. The machine learning model 502 may be configured to perform pixel quantification of a container. The container may be located in a containerized environment associated with inventory. The image of the container includes pixels that correspond to objects (e.g., elements or components) within the image. For example, an image of the container may contain pixels of the image corresponding to the container, pixels of the image corresponding to the inventory item, and, in some implementations, pixels of the image corresponding to a label for the inventory item. The image of the container may also include pixels of the image corresponding to areas surrounding the container, for example, another container or the containerized environment.

Pixel quantification may be used to estimate inventory volume in the container, for example, by calculating a proportion of the number of inventory item pixels relative to the number of container pixels. For example, an image of an empty container may include approximately 100% of pixels corresponding to the container and approximately 0% of pixels corresponding to the inventory item. In another example, an image of a partially full container may include approximately 80% of pixels corresponding to the container and approximately 20% of pixels corresponding to the inventory item. In another example, an image of a full container may include approximately 50% of pixels corresponding to the container and approximately 50% of pixels corresponding to the inventory item. Pixel quantification may use a proportion (e.g. ratio or percentage) of pixels corresponding to different objects in the image such that images captured at different distances from the container may be used to accurately estimate inventory volume.

The imaging device may be positioned facing or above the container, with pixel quantification varying by placement. For an image facing the container, pixel quantification calculates the proportion of inventory item pixels to container wall pixels, often requiring items to be pushed back for visibility. For an image above the container, it uses the proportion of inventory item pixels to container floor pixels, allowing items to be placed anywhere within the container for more accurate fill status estimation.

The machine learning model 502 may be trained using training data 504. The training data 504 may include a set of images. Each image may depict a container in a known state (e.g., full, partially full, or empty). The set of images may be captured specifically for training the machine learning model 502. Alternatively, the set of images may be captured by installing an imaging device to capture images of the container over time. The set of images may include, for example, three images, ten images, thirty images, one hundred images, two hundred images, five hundred images, one thousand images, or more. Each image may be labeled with the known state for the container (e.g., a set of labeled images). The set of images may be labeled by a user of the intelligent vision system. Alternatively, labels for the set of images may be generated by, for example, a machine learning model trained to label images. Each image may include annotations that identify pixel-level classifications used to associate pixels with objects within the image. The pixel-level classifications may include, for example, color, shape, size, or spatial proximity to known reference features. For example, pixels corresponding to the inventory item may be associated with a specific color or a location relative to the container. In another example, pixels associated with the container may be associated with a specific color, a shape of the container, or a known size of the container within the image.

After training, the machine learning model 502 may be applied to a new image 506. The new image 506 may be captured by an imaging device (e.g., the imaging device 404 or the imaging and tracking device 200). The new image 506 may include at least one container in an unknown state. The new image 506 may be acquired in response to a triggering condition. The machine learning model 502 may segment pixels of the new image 506 and classify at least some of the pixels as pixels corresponding to the container (e.g., container pixels) and at least some of the pixels as pixels corresponding to the inventory item (e.g., inventory item pixels). The machine learning model 502 may perform pixel quantification on the new image 506. For example, the machine learning model 502 may calculate a proportion of the number of inventory item pixels relative to the number of container pixels. The machine learning model 502 may account for known reference features in the new image 506, for example, printed markings or labels.

The output of the machine learning model 502 may be an inference 508 that indicates a status of the container. The inference 508 may be generated using a regression algorithm. The status may include discrete categories such as full, partially full, or empty, or a continuous value representing a fill level. The inference 508 may also include a confidence score indicating the likelihood of accuracy for the prediction. In some implementations, the inference 508 may be transmitted to an inventory management system or user interface for further processing, display, or action. The inference 508 may trigger automated logic such as generating alerts or initiating a reorder sequence if the status of the container falls below a defined threshold.

In some implementations, the machine learning model 502 may be periodically or continuously retrained. The machine learning model 502 may be retrained using an updated training data set. The updated training set may include newly labeled images. In an example, the newly labeled images may reflect changes in container layouts, lighting conditions, inventory types, or visual markers. Retraining the machine learning model 502 may improve accuracy over time and allow the system to adapt to dynamic conditions in health care environments. The retraining process may be performed manually by an operator or may occur automatically based on predefined triggers or performance thresholds. For example, the system may monitor prediction confidence scores or user feedback and initiate retraining when confidence falls below a specified value. The retraining process may use the same architecture as the original model or apply updated parameters adjust the machine learning model 502.

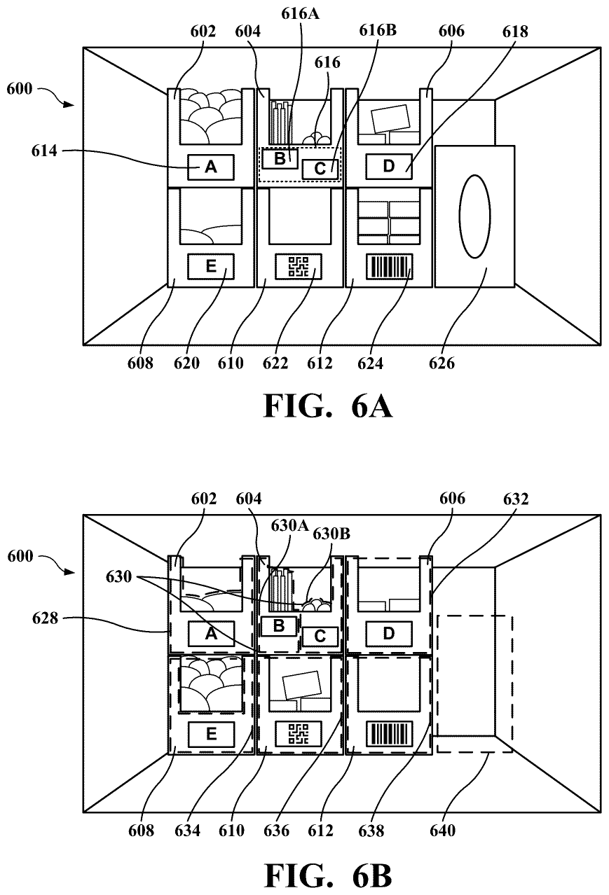

FIGS. 6A and 6B illustrate an example of a containerized environment 600. The containerized environment 600 may be associated with health care inventory management. An imaging device (e.g., the imaging and tracking device 200 shown in FIG. 2) is positioned to capture images of the containerized environment 600. The imaging device may be positioned above or to a side of the containerized environment 600. The imaging device may capture images of the containerized environment 600 from an overhead or angled view. The imaging device may capture images of multiple containers in a single field of view. The imaging device may be configured to capture images of the containerized environment 600 in response to detecting a triggering condition.

FIG. 6A shows containers within the containerized environment 600 (e.g., a first container 602, a second container 604, a third container 606, a fourth container 608, a fifth container 610, and a sixth container 612). The containers are housed within a furniture unit (e.g., the furniture unit 104), which may include a cabinet, shelving system, or supply cart used in clinical environments. Each container may include (e.g., hold) one or more inventory items. Each inventory item may include (e.g., exhibit or be associated with) different visual characteristics, for example, volume (e.g., fill level), color, shape, size, spatial proximity to known features of a container containing the inventory item, or arrangement (e.g., a particular way an inventory item is stored in the container, for example, stacked boxes). Containers may include markings covered by inventory items when the fill level is sufficient and more visible when the fill level is insufficient (e.g., low).

A container may include a label (i.e., a first label 614 on the first container 602, a second label 616 on the second container 604, a third label 618 on the third container 606, a fourth label 620 on the fourth container 608, a fifth label 622 on the fifth container 610, and a sixth label 624 on the sixth container 612). The label may be divided into two or more parts, for example, to signify that more than one inventory item is stored in a same container (e.g., the second label 616 is divided into a first label part 616A and a second label part 616B). The label may be readable using optical character recognition (OCR) technology.

For example, the label may include alphanumeric characters (e.g., the first label 614 contains a letter “A,” the first label part 616A of the second label 616 contains a letter “B,” the second label part 616B of the second label 616 contains a letter “C,” the third label 618 contains a letter “D,” and the fourth label 620 contains an “E”). Alternatively, the label may contain a computer-readable (e.g., scannable) image, for example, a Quick Response (QR) code (e.g., the fifth label 622), a barcode (e.g., the sixth label 624), or the like. The label may be facing the imaging device. The labels may be located within or near the containers and may be captured as part of the image. The labels may be used by the intelligent vision system to classify containers and associate containers with specific inventory types.

FIG. 6A represents an image as captured by the imaging device. The image shown in FIG. 6A may be used as a training image for a machine learning model (e.g., the machine learning model 502). Alternatively, the image shown in FIG. 6A may be used as a new image for a machine learning model (e.g., the machine learning model 502). Pixel quantification may be performed to determine a status of the containers shown in the image, for example, to determine whether the container is full, partially full, or empty. The image includes one or more containers, their contents (e.g., inventory items), and any visible visual indicators (e.g., labels), without bounding boxes or segmentation overlays.

FIG. 6A also shows an inventory item 626 that is not stored in a container. The inventory item 626 may be stored directly on the furniture unit, that is, the furniture unit (e.g., a portion of the furniture unit) may be considered a container for the inventory item 626. For an inventory item not stored in a container, the machine learning model (e.g., the machine learning model 502) may be trained to classify pixels as pixels corresponding to the inventory item or pixels corresponding to the furniture unit. The furniture unit may contain markings used to determine whether an inventory item 626 is in stock. For example, the furniture unit may include a number of markings indicating a number of the inventory item 626 that are desired to be in stock. Each marking visible within an image indicates an inventory item 626 that is missing (e.g., out of stock).

FIG. 6B shows an image of the containers depicted in FIG. 6A, but further includes bounding boxes and segmentation overlays. FIG. 6B may represent an annotated training image, for example, with bounding boxes and annotations provided by a user. The bounding boxes may be drawn by the user or may be generated, for example, using computer vision techniques. The bounding boxes may be verified or adjusted by the user prior to using the image as a training image. The annotations may be stored as metadata within the annotated training image. Alternatively, FIG. 6B may represent a new image that has been processed by the intelligent vision system to generate bounding boxes. Each container may have one or more bounding boxes (i.e., a first bounding box 628 for the first container 602, a second bounding box 630 for the second container 604, a third bounding box 632 for the third container 606, a fourth bounding box 634 for the fourth container 608, a fifth bounding box 636 for the fifth container 610, and a sixth bounding box 638 for the sixth container 612). The bounding boxes may surround the containers within the image. Each inventory item may be associated with a bounding box, such that containers with multiple inventory items may have multiple bounding boxes (e.g., the second container 604 may have a first bounding box part 630A and a second bounding box part 630B). The bounding boxes define the boundaries of each container. The bounding boxes may include visual classification of internal regions based on pixel categorization. The bounding boxes may be used to support confidence scoring or further inference about restocking needs.

The bounding boxes may surround (e.g., include or encompass) a container. In some implementations, a bounding box may be segmented to exclude an area corresponding to, for example, a background, the furniture unit, or the like. For example, the first bounding box 628 has been segmented to include the first container 602 and an associated inventory item, but to exclude a ceiling and back wall of the furniture unit. Accordingly, for pixel quantification of the first bounding box 628, only pixels corresponding to the first container 602 and the associated inventory item may be considered. In other implementations, a bounding box may be segmented to divide an area corresponding to the container and an area corresponding to the associated inventory item. For example, the fourth bounding box 634 has been segmented to include a first area corresponding to the fourth container 608 and a second area corresponding to the associated inventory item. Accordingly, for pixel quantification of the fourth bounding box 634, a number of pixels in the first area corresponding to the fourth container 608 and a number of pixels in the second area corresponding to the associated inventory item may be used to estimate a volume of the associated inventory item.

FIG. 6B also shows a seventh bounding box 640 corresponding to a location of the inventory item 626. The inventory item 626 is not present in FIG. 6B. Accordingly, the machine learning model (e.g., the machine learning model 502) may classify substantially all pixels within the seventh bounding box 640 as pixels corresponding to the furniture unit. The seventh bounding box 640 may be generated based on object edges, spatial clustering, or prior training data.

While the disclosure primarily describes bounding boxes for defining regions around containers, the intelligent vision system is not limited to such regions. In some implementations, the system may identify contours or segmentation regions to enclose inventory items with cylindrical or round shapes, enhancing precision over rectangular bounding boxes. Similarly, for containers with irregular or non-rectangular shapes, the system may define customized segmentation regions based on visual features or training data to enable accurate pixel quantification.

In some embodiments, the intelligent vision system may detect a label within the image and classify a container associated with the label as a container corresponding to an inventory item based on the label. Label detection may use, for example, OCR, barcode scanning, or QR code recognition to extract data and associate it with an inventory item. A list of inventory items may be stored in a digital catalog. The label may be associated with a container based on the label being located within a bounding box associated with the container. Labels may be treated as pixel regions that are separately categorized from inventory item pixels and container pixels.

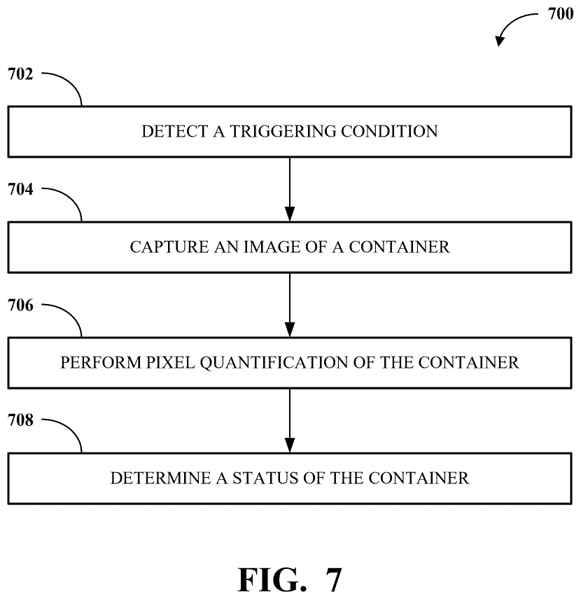

FIG. 7 is a flowchart showing an example of a first technique 700 for determining inventory using an intelligent vision system. The first technique 700 can be executed using computing devices, such as the systems, hardware, and software described with respect to FIGS. 1-5. The first technique 700 can be performed, for example, by executing a machine-readable program or other computer-executable instructions, such as routines, instructions, programs, or other code. The steps, or operations, of the first technique 700 or another technique, method, process, or algorithm described in connection with the implementations disclosed herein can be implemented directly in hardware, firmware, software executed by hardware, circuitry, or a combination thereof.

At 702, a triggering condition is detected by an imaging device positions to capture images of one or more containers. The triggering condition may be detected by an imaging device. The imaging device may be positioned to capture an image of a containerized environment, for example, a containerized environment associated with health care inventory. The triggering condition may include motion detection, such as when a person reaches into or interacts with a furniture unit (e.g., cabinet, drawer, shelf, or the like). In some cases, the system may detect vibration using a sensor that senses activity within the furniture unit, such as the opening of a drawer or the removal of an item. Additionally, the triggering condition may include a sensor measurement, such as vibration intensity or frequency, exceeding a predefined threshold, causing the imaging device to transition from a wait state, where image capture is restricted to preserve resources, to an active state, enabling capture of one or more images of inventory containers within the furniture unit. Alternatively, the triggering condition may include expiration of a predefined time interval, such as every 30 minutes, every three hours, every shift, or on a fixed schedule. Detecting the triggering condition may activate an imaging device positioned to capture a containerized environment in which one or more health care inventory containers are stored.

At 704, the imaging device captures an image of a container. The image may be captured in response to the triggering condition. The image may be a still image or a frame from a video feed. The container may be a bin, shelf compartment, drawer segment, or similar structure. The container may include one or more inventory items. The image may also include visual indicators, such as printed product labels, QR codes, or barcodes. The visual indicators may be used to classify or associate the container with a specific inventory item.

At 706, the system performs pixel quantification of the container using a trained machine learning model. The model may be trained using a labeled training dataset that includes images of the containerized environment or containers in known fill states, such as fully stocked, half full, and empty. During inference, the system may segment pixels of the image of the container to identify pixels that correspond to inventory items (e.g., inventory item pixels) and pixels that correspond to the container (e.g., container pixels). The system may determine a number of inventory item pixels and a number of container pixels. Some pixels may also be classified as label pixels, for example, when the system identifies product names, text, or standardized visual markers. Pixel classification may be based on color gradients (e.g., distinguishing white gauze from a beige bin), object shapes (e.g., the cylindrical profile of syringes), size (e.g., a size of the inventory item compared to a size of the container), and spatial proximity to known features of the container (e.g., container walls or a label on the container).

At 708, the system determines a status of the container based on the pixel quantification. The status of the container may be, for example, a fill status. The fill status may be expressed as a category (e.g., full, partially full, or empty) or as a percentage estimate of volume occupied by inventory items. For example, if 35% of the container pixels are classified as inventory item pixels, the system may determine that the fill status is 50% full. The machine learning model may also assign a confidence score to this estimate, such as a 90% confidence that the container is between 40-60% full. The status of the container may be determined by calculating a proportion of the number of inventory item pixels relative to the number of container pixels. In some implementations, the system may extract label pixels and use optical character recognition (OCR) to associate the label with a specific inventory item, for example, “IV bag” or “glucose test strips.”

The system may further generate an alert when the determined fill status falls below a predefined threshold, such as when a container is less than 50% full. This alert may be transmitted to a user device as a push notification, text message, or interface banner. In some cases, the alert may also include the item name and location of the container. Additionally, the system may transmit the fill status to an inventory management system for storage, display, or order initiation. For example, the system may automatically generate a restock request for the container when an alert is triggered. In another example, the system may automatically place a purchase order for the inventory item associated with the container.

The first technique 700 may further include configuring the imaging device. Configuring the imaging device may include coupling at least a portion of the imaging device to a portion of the furniture unit. The imaging device can be removably coupled to the furniture unit, for example, to enable easy relocation of the imaging device. Alternatively, the imaging device can be permanently coupled to the furniture unit. In some implementations, configuring the imaging device further includes adjusting a field of view of an image sensor of the imaging device to enable an image sensor to capture an image including each container. Adjusting the field of view of the image sensor can include remotely controlling a pan/tilt motor of the image sensor to adjust an orientation of the image sensor. In some implementations, the remote controlling of the pan/tilt motor can be automated by the software application. For example, the software application can automatically adjust the orientation of the image sensor responsive to a determination that a field of view of the image sensor does not capture all or a threshold number of containers stored within a furniture unit.

The first technique 700 may include automatically updating a database record associated with the container or the inventory item based on the status of the container. The database record may be secured to a private cloud or local servers. The database record may include automatic backup sync such that the database record does not require internet connectivity at all times. Updating the database record may occur without manual user intervention. The system may receive an indication of the inventory item associated with the container. The system may query a database using an identifier of the inventory item to retrieve a database record associated with the inventory item from the database. The system may then update the database record. Updating the database record may include updating one or more pieces of data included in the database record. For example, the status of the container may be used to update data of the database record including, but not limited to, a status of the container, an estimated volume of the inventory item, a total inventory number for the inventory item, a time at which the status was determined, a time at which the database record was updated, an indication of the container or the furniture unit, a task created for performance using the inventory item, or other information.

The first technique 700 may further include transmitting, by the system, an update to a client device. The update may include information, instructions, or the like and is used to indicate a user of the client device as to the updated database record. For example, the update may include instructions for rendering a GUI at the client device. The GUI may include updated inventory information for the container or inventory item. In some implementations, the update may be an alert (e.g., a push notification, text message, or other alert) indicating a task to perform associated with the inventory item.

In some such implementations, the first technique 700 may include changing a state of the imaging device from a wait state to an active state. For example, upon detecting the triggering condition, the processor may change the state of the imaging device from a wait state to an active state, such as to enable the use of the image sensor. In some such implementations, the processor changes the state of the imaging device in response to a determination that the vibration detected using the accelerometer or other vibration sensor meets a threshold. For example, the threshold may be used to prevent false positive situations in which the furniture unit is exposed to a vibration unrelated to the accessing of the furniture unit.

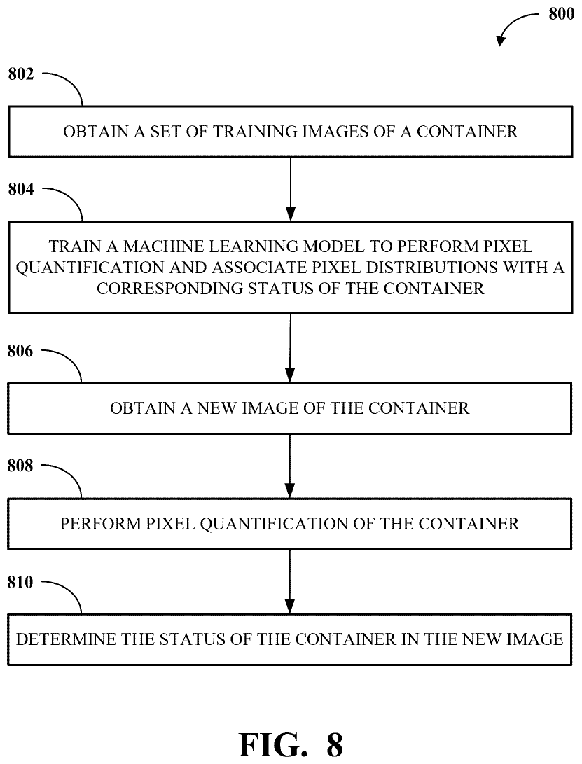

FIG. 8 is a flowchart showing an example of a second technique 800 for training and applying a machine learning model to determine a fill status of a container. The second technique 800 can be executed using computing devices, such as the systems, hardware, and software described with respect to FIGS. 1-5. The second technique 800 can be performed, for example, by executing a machine-readable program or other computer-executable instructions, such as routines, instructions, programs, or other code. The steps, or operations, of the second technique 800 or another technique, method, process, or algorithm described in connection with the implementations disclosed herein can be implemented directly in hardware, firmware, software executed by hardware, circuitry, or a combination thereof.

At 802, a set of training images of a container are obtained. The training images may be images of the containerized environment (e.g., the containerized environment 600 shown in FIG. 6A). The training images may depict the container in a variety of known fill states. For example, the training images may include at least one image showing the container as each of full, partially full, and empty. Each training image may include labeled pixel data identifying inventory item pixels and container pixels. The labeled pixel data may include pixels labeled as label pixels corresponding to a product label on or near the container. The training images may also include annotations for visual characteristics such as color, shape, size, or proximity to known features, providing a basis for learning pixel-level distinctions. The labeled training images may be manually labeled or generated using semi-automated or automated tools.

At 804, a machine learning model is trained to perform pixel quantification and associate pixel distributions with a corresponding fill status of the container. The training process may use a regression algorithm or a classification architecture such as a convolutional neural network. The machine learning model may be trained to identify patterns in pixel distribution that correlate with container volume. For example, the model may learn that in an empty container, a proportion of the number of inventory item pixels relative to the number of container pixels is small, whereas in a full container, a greater percentage of pixels are identified as inventory item pixels.

At 806, a new image of the container is obtained. The new image may be an image of the containerized environment (e.g., the containerized environment 600 shown in FIG. 6A). The new image may be captured in real time by the imaging device. The new image may reflect current inventory conditions and may include visual indicators such as printed labels, QR codes, or barcodes. The new image may be used for inference and may differ from the training images in lighting, angle, or container contents.

At 808, pixel quantification of the container in the new image is performed using the trained machine learning model. The system may segment the container into pixel regions and classify pixels into one or more categories. Pixel quantification may include instance segmentation. For example, an instance segmentation algorithm may be applied to the container in the new image to separate pixels associated with an inventory item from pixels associated with the container. Pixel quantification may include generating a bounding box within the new image, for example, a bounding box surrounding the container. Classification of pixels may rely on visual features such as color tone (e.g., identifying white gauze versus a dark plastic bin), shape (e.g., cylindrical objects), and spatial relationships.

At 810, the system determines the status of the container in the new image. The status may be output as a categorical label (e.g., full, partially full, or empty), or a numeric value (e.g., 63% full). In some implementations, the system may generate a confidence score associated with the determined status. The machine learning model may also assign a confidence score to this estimate, such as a 90% confidence that the container is between 60-65% full. The status of the container may be determined by calculating a proportion of the number of inventory item pixels relative to the number of container pixels.

In some implementations, the system may identify one or more product labels in the image and classify the container accordingly. This may include classifying label pixels and applying OCR to associate the container with a specific inventory item. The system may generate an alert if the fill status falls below a threshold and may automatically place a purchase order for the corresponding inventory item.

For simplicity of explanation, the first technique 700 and the second technique 800 are each depicted and described herein as a respective series of steps or operations. However, the steps or operations in accordance with this disclosure can occur in various orders and/or concurrently. Additionally, other steps or operations not presented and described herein may be used. Furthermore, not all illustrated steps or operations may be required to implement a technique in accordance with the disclosed subject matter.

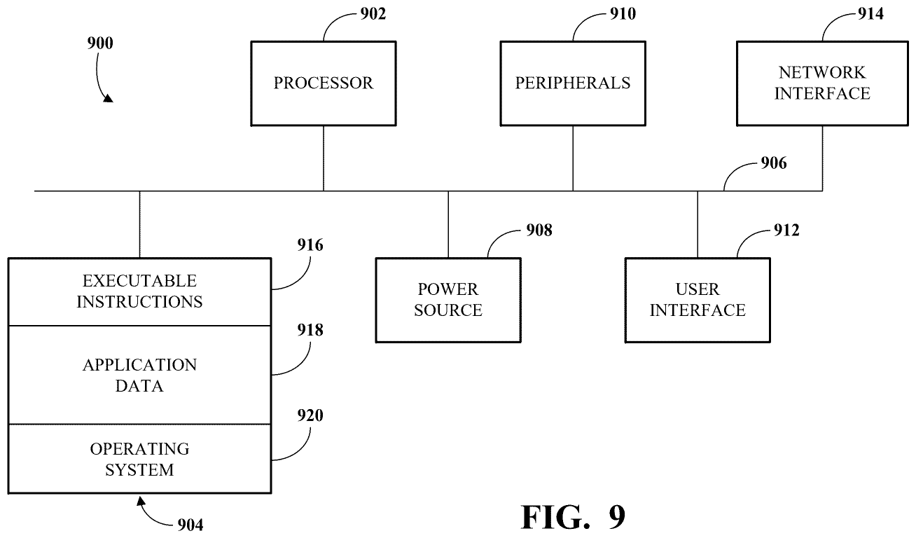

FIG. 9 is a block diagram showing an example of a computing device 900 which may be used in a real-time health care inventory imaging and tracking intelligence system, for example, the system 100 shown in FIG. 1. The computing device 900 may be used to implement a server on which a software application is run (e.g., the server 106 and the software application 108 shown in FIG. 1). Alternatively, the computing device 900 may be used to implement a client that accesses the software application (e.g., the client 116 shown in FIG. 1). As a further alternative, the computing device 900 may be used as or to implement another client, server, or other device according to the implementations disclosed herein. The computing device 900 includes components or units, such as a processor 902, a memory 904, a bus 906, a power source 908, peripherals 910, a user interface 912, and a network interface 914. One or more of the memory 904, the power source 908, the peripherals 910, the user interface 912, or the network interface 914 can communicate with the processor 902 via the bus 906.

The processor 902 is a central processing unit, such as a microprocessor, and can include single or multiple processors having single or multiple processing cores. Alternatively, the processor 902 can include another type of device, or multiple devices, now existing or hereafter developed, configured for manipulating or processing information. For example, the processor 902 can include multiple processors interconnected in any manner, including hardwired or networked, including wirelessly networked. For example, the operations of the processor 902 can be distributed across multiple devices or units that can be coupled directly or across a local area or other suitable type of network. The processor 902 can include a cache, or cache memory, for local storage of operating data and/or instructions.

The memory 904 includes one or more memory components, which may each be volatile memory or non-volatile memory. For example, the volatile memory of the memory 904 can be random access memory (RAM) (e.g., a DRAM module, such as DDR SDRAM) or another form of volatile memory. In another example, the non-volatile memory of the memory 904 can be a disk drive, a solid state drive, flash memory, phase-change memory, or another form of non-volatile (or non-transitory) memory configured for persistent electronic information storage. The memory 904 may also include other types of devices, now existing or hereafter developed, configured for storing data or instructions for processing by the processor 902.

The memory 904 can include data for immediate access by the processor 902. For example, the memory 904 can include executable instructions 916, application data 918, and an operating system 920. The executable instructions 916 can include one or more application programs, which can be loaded or copied, in whole or in part, from non-volatile memory to volatile memory to be executed by the processor 902. For example, the executable instructions 916 can include instructions for performing some or all of the techniques of this disclosure. The application data 918 can include user data, database data (e.g., database catalogs or dictionaries), or the like. The operating system 920 can be, for example, Microsoft Windows®, Mac OS X®, or Linux®; an operating system for a small device, such as a smartphone or tablet device; or an operating system for a large device, such as a mainframe computer.

The power source 908 includes a source for providing power to the computing device 900. For example, the power source 908 can be an interface to an external power distribution system. In another example, the power source 908 can be a battery, such as where the computing device 900 is a mobile device or is otherwise configured to operate independently of an external power distribution system.

The peripherals 910 includes one or more sensors, detectors, or other devices configured for monitoring the computing device 900 or the environment around the computing device 900. For example, the peripherals 910 can include a geolocation component, such as a global positioning system location unit. In another example, the peripherals can include a temperature sensor for measuring temperatures of components of the computing device 900, such as the processor 902.

The user interface 912 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 914 provides a connection or link to a network (e.g., the network 114 shown in FIG. 1). The network interface 914 can be a wired network interface or a wireless network interface. The computing device 900 can communicate with other devices via the network interface 914 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

Implementations of the computing device 900 may differ from what is shown and described with respect to FIG. 9. In some implementations, the computing device 900 can omit the peripherals 910. In some implementations, the memory 904 can be distributed across multiple devices. For example, the memory 904 can include network-based memory or memory in multiple clients or servers performing the operations of those multiple devices. In some implementations, the application data 918 can include functional programs, such as a web browser, a web server, a database server, another program, or a combination thereof.

In some implementations, the intelligent vision system may be positioned above or in front of pharmacy shelves to capture images of medication bottles, boxes, or blister packs stored in designated locations. Using pixel quantification, the system may estimate the fill status of containers, determine which medications are running low, and identify when restocking may be needed. In automated pharmacy settings, such as robotic dispensing systems, the system may estimate the volume of loose pills or capsules remaining in large dispensing canisters or hoppers. By analyzing the surface level or pixel density of medication within each container, the system may generate fill-level estimates and predict when a refill is required. Additionally, the system may classify inventory based on label features or visual indicators, enabling traceability of specific drug types and dosage forms.

In some implementations, the intelligent vision system may be implemented in surgical supply rooms to monitor sterile inventory such as gauze, sutures, syringes, scalpels, and procedure-specific kits. Containers holding these items may be imaged periodically or upon detection of a triggering condition, such as movement of the supply bin. Pixel quantification may allow the system to detect changes in volume that result from item usage, supporting continuous inventory updates without requiring staff intervention. The system may also detect product labels or visual markers to differentiate between sterile supply types or usage categories.

In some implementations, the intelligent vision system may be implemented in manufacturing or industrial settings to monitor the volume of parts or materials. For example, a container of manufacturing materials may be imaged at set intervals or upon sensing movement. The system may estimate the fill status of the container using pixel quantification and trigger a restocking alert when inventory falls below a set threshold.

In some implementations, the intelligent vision system may be used to monitor stock levels on shelves or within backroom containers for retail products. The intelligent vision system may use images of display units or storage bins and automatically assess product volume to alert when items may need to be restocked. The intelligent vision system may recognize product labels or packaging to distinguish between different SKUs and may associate fill levels with specific inventory items.

The implementations of this disclosure describe methods, systems, devices, apparatuses, and non-transitory computer-readable media for implementing an intelligent vision system to perform pixel quantification of a container.

Clause 1. A method, comprising: detecting a triggering condition by an imaging device; capturing, by the imaging device in response to the triggering condition, an image of a container; performing, by a machine learning model, pixel quantification of the container; and determining, based on the pixel quantification, a status of the container.

Clause 2. The method of clause 1, wherein performing the pixel quantification of the container comprises: segmenting pixels of the image of the container, wherein at least some of the pixels are classified as inventory item pixels or container pixels; and determining a number of inventory item pixels and a number of container pixels; and determining the status of the container comprises: calculating a proportion of the number of inventory item pixels relative to the number of container pixels.

Clause 3. The method of clause 2, wherein the inventory item pixels are based on one or more visual characteristics comprising at least one of color, shape, size, or spatial proximity to known reference features.

Clause 4. The method of clause 2, further comprising: detecting a product label within the image, wherein at least some of the pixels are classified as label pixels; and classifying the container as corresponding to an inventory item based on the product label.

Clause 5. The method of clause 1, further comprising: generating an alert when the status of the container corresponds to a predefined threshold condition.

Clause 6. The method of clause 1, further comprising: transmitting the status of the container to an inventory management system for storage, display, or order initiation.

Clause 7. The method of clause 1, wherein the triggering condition comprises motion detection or vibration detection.

Clause 8. The method of clause 1, wherein the triggering condition comprises expiration of a predefined time interval.

Clause 9. The method of clause 1, wherein the status of the container is full, partially full, or empty.

Clause 10. A system, comprising: an imaging device configured to: detect a triggering condition; and capture, in response to the triggering condition, an image of a container; and a server device in communication with the imaging device, wherein the server device comprises a processor configured to: receive, from the imaging device, the image of the container; perform, by a machine learning model, pixel quantification of the container; and determine, based on the pixel quantification, a status of the container.

Clause 11. The system of clause 10, wherein to perform the pixel quantification of the container comprises to: segment pixels of the image of the container, wherein at least some of the pixels are classified as inventory item pixels or container pixels; and determining a number of inventory item pixels and a number of container pixels; and to determine the status of the container comprises to: calculate a proportion of the number of inventory item pixels relative to the number of container pixels.

Clause 12. The system of clause 11, wherein the processor is further configured to: detect a product label within the image, wherein at least some of the pixels are classified as label pixels; and classify the container as corresponding to an inventory item based on the product label.

Clause 13. The system of clause 10, wherein the processor is further configured to: generate, based on the status of the container, an alert; and transmit the alert to a user device.

Clause 14. The system of clause 10, wherein the imaging device is further configured to: receive, from the server device, an updated triggering condition; replace the triggering condition with the updated triggering condition; and transmit, to the server device, confirmation of the updated triggering condition.

Clause 15. A method, comprising: obtaining a set of training images comprising images of a container associated with inventory, wherein each training image is labeled with a status of the container; training, using the set of training images, a machine learning model to perform pixel quantification and associate pixel distributions with a corresponding status of the container; obtaining a new image of the container; performing, by the machine learning model for the container in the new image, pixel quantification of the container; and determining, by the machine learning model, the status of the container in the new image.

Clause 16. The method of clause 15, wherein performing the pixel quantification of the container comprises: segmenting pixels of the new image of the container, wherein at least some of the pixels are classified as inventory item pixels or container pixels; and determining a number of inventory item pixels and a number of container pixels; and determining the status of the container comprises: calculating a proportion of the number of inventory item pixels relative to the number of container pixels.

Clause 17. The method of clause 15, wherein performing pixel quantification of the container comprises: applying an instance segmentation algorithm to the container to separate pixels associated with an inventory item from pixels associated with the container.

Clause 18. The method of clause 15, further comprising: identifying one or more visual characteristics in the new image, wherein the one or more visual characteristics comprise at least one of color, shape, size, or spatial proximity to known reference features; and associating the one or more visual characteristics with an inventory item corresponding to the container.

Clause 19. The method of clause 15, wherein determining the status comprises: generating a confidence score associated with the status.

Clause 20. The method of clause 15, further comprising: generating, based on the status of the container, an inventory alert for an inventory item associated with the container; and placing, automatically based on the inventory alert, a purchase order for the inventory item.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc.

Likewise, the terms “system” or “mechanism” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

Implementations or portions of implementations of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method, comprising:
detecting a triggering condition by an imaging device;
capturing, by the imaging device in response to the triggering condition, an image of a container;
performing, by a machine learning model, pixel quantification of the container; and
determining, based on the pixel quantification, a status of the container.

2. The method of claim 1, wherein
performing the pixel quantification of the container comprises:
segmenting pixels of the image of the container, wherein at least some of the pixels are classified as inventory item pixels or container pixels; and
determining a number of inventory item pixels and a number of container pixels;
and
determining the status of the container comprises:
calculating a proportion of the number of inventory item pixels relative to the number of container pixels.

3. The method of claim 2, wherein the inventory item pixels are based on one or more visual characteristics comprising at least one of color, shape, size, or spatial proximity to known reference features.

4. The method of claim 2, further comprising:
detecting a product label within the image, wherein at least some of the pixels are classified as label pixels; and
classifying the container as corresponding to an inventory item based on the product label.

5. The method of claim 1, further comprising:
generating an alert when the status of the container corresponds to a predefined threshold condition.

6. The method of claim 1, further comprising:
transmitting the status of the container to an inventory management system for storage, display, or order initiation.

7. The method of claim 1, wherein the triggering condition comprises motion detection or vibration detection.

8. The method of claim 1, wherein the triggering condition comprises expiration of a predefined time interval.

9. The method of claim 1, wherein the status of the container is full, partially full, or empty.

10. A system, comprising:
an imaging device configured to:
detect a triggering condition; and
capture, in response to the triggering condition, an image of a container; and
a server device in communication with the imaging device, wherein the server device comprises a processor configured to:
receive, from the imaging device, the image of the container;
perform, by a machine learning model, pixel quantification of the container; and
determine, based on the pixel quantification, a status of the container.

11. The system of claim 10, wherein
to perform the pixel quantification of the container comprises to:
segment pixels of the image of the container, wherein at least some of the pixels are classified as inventory item pixels or container pixels; and
determining a number of inventory item pixels and a number of container pixels;
and
to determine the status of the container comprises to:
calculate a proportion of the number of inventory item pixels relative to the number of container pixels.

12. The system of claim 11, wherein the processor is further configured to:
detect a product label within the image, wherein at least some of the pixels are classified as label pixels; and
classify the container as corresponding to an inventory item based on the product label.

13. The system of claim 10, wherein the processor is further configured to:
generate, based on the status of the container, an alert; and
transmit the alert to a user device.

14. The system of claim 10, wherein the imaging device is further configured to:
receive, from the server device, an updated triggering condition;
replace the triggering condition with the updated triggering condition; and
transmit, to the server device, confirmation of the updated triggering condition.

15. A method, comprising:
obtaining a set of training images comprising images of a container associated with inventory, wherein each training image is labeled with a status of the container;
training, using the set of training images, a machine learning model to perform pixel quantification and associate pixel distributions with a corresponding status of the container;
obtaining a new image of the container;
performing, by the machine learning model for the container in the new image, pixel quantification of the container; and
determining, by the machine learning model, the status of the container in the new image.

16. The method of claim 15, wherein
performing the pixel quantification of the container comprises:
segmenting pixels of the new image of the container, wherein at least some of the pixels are classified as inventory item pixels or container pixels; and
determining a number of inventory item pixels and a number of container pixels;
and
determining the status of the container comprises:
calculating a proportion of the number of inventory item pixels relative to the number of container pixels.

17. The method of claim 15, wherein performing pixel quantification of the container comprises:
applying an instance segmentation algorithm to the container to separate pixels associated with an inventory item from pixels associated with the container.

18. The method of claim 15, further comprising:
identifying one or more visual characteristics in the new image, wherein the one or more visual characteristics comprise at least one of color, shape, size, or spatial proximity to known reference features; and
associating the one or more visual characteristics with an inventory item corresponding to the container.

19. The method of claim 15, wherein determining the status comprises:
generating a confidence score associated with the status.

20. The method of claim 15, further comprising:
generating, based on the status of the container, an inventory alert for an inventory item associated with the container; and
placing, automatically based on the inventory alert, a purchase order for the inventory item.
